(19) United States
(12) Patent Application Publication
(10) Pub. No.: US 2009/0204078 A1
(43) Pub. Date: Aug. 13, 2009

(54) Manifold and Valve Seal for Use with a Medical Device

(75) Inventors: James J. Mitchell, Ballston Spa, NY (US); Mark H. Van Diver, Argyle, NY (US); Scott A. Diamond, Glens Falls, NY (US); Colin P. Hart, Queensbury, NY (US); Waleed El Sayyid, Clifton Park, NY (US)

Correspondence Address: ORRICK, HERRINGTON & SUTCLIFFE, LLP;IP PROSECUTION DEPARTMENT, 4 PARK PLAZA, SUITE 1600, IRVINE, CA, 92614-2558, US

(73) Assignee: BOSTON SCIENTIFIC SCIMED, INC., Maple Grove, MN (US)

(21) Appl. No.: 12/030,271

(22) Filed: Feb. 13, 2008

(51) Int. Cl. A61M 5/00

(52) U.S. Cl. 604/246

(57) ABSTRACT

Disclosed is a manifold including a housing having multiple ports and multiple bores networking the ports. An actuatable valve, such as a slidable valve, including one or more bores may be positioned in the housing such that the valve is actuatable between a first position and a second position. Actuation of the valve may selectively establish a desired fluid pathway through the manifold. One or more seals may be interposed between the valve and the housing. The seal includes at least a first sealing element, a second sealing element, and a land extending between the sealing elements. The sealing elements are configured to provide a fluid tight seal between the housing and the valve when the valve is in either the first or second position, and the land is configured to provide a fluid tight seal between the housing and the valve when the valve is in an intermediate position.

### TECHNICAL FIELD

The invention generally relates to the area of medical devices. More particularly, the invention relates to manifolds having multiple ports for coupling to medical apparatuses and/or multiple bores for controlling fluid pathways during a medical procedure.

### BACKGROUND

Various medical procedures involve the introduction of fluids into the body of a patient and/or the removal of fluids from the body of a patient. A variety of manifolds have been developed which selectively control fluids being passed into and out of the body of a patient. Some such manifolds are configured to couple multiple medical apparatuses and/or fluid sources with one or more medical devices. In some medical procedures, it may be desirous that the manifold be capable of withstanding pressures in excess of 100 psi, 300 psi, or 500 psi or more. However, fluid leakage or bypass within and/or egressing from the manifold may be experienced at some operative pressures. Known manifolds require close dimensional tolerances to be maintained in order to prevent fluid leakage or bypass.

It may be desirous to provide a manifold having an actuatable valve for selectively controlling fluid pathways through the manifold. Furthermore, it may be desirous to prevent fluid leakage or bypass at the interface between the actuatable valve and the housing of the manifold, yet desirously maintaining a modest actuation force necessary to actuate the valve.

### SUMMARY

The invention provides several alternative designs, materials, and applications of a sealing member and/or a manifold for use in a medical procedure.

Accordingly, one embodiment of an exemplary seal includes a primary sealing portion, which may include one or more, for example, one, two or three, sealing elements, such as annular sealing elements. Additionally, the exemplary seal includes a secondary sealing portion, which may include one or more, for example one, two or three, bands of material, such as lands, extending from and/or between the sealing element(s).

One embodiment of an exemplary manifold includes a housing having a plurality of ports defined therein. The housing may additionally include a plurality of bores in fluid communication with one or more of the plurality of ports. An actuatable valve, which may be selectively actuated to establish a desired fluid pathway through the manifold, may be positioned in the housing. A seal may be interposed between the actuatable valve and the housing to prevent fluid leakage or bypass at the interface between the actuatable valve and the housing.

Another embodiment includes a manifold including a housing having a plurality of ports and a plurality of bores networking the plurality of ports. An actuatable valve including one or more bores may be positioned in the housing. The actuatable valve may be actuatable between a first position and a second position, such that actuation of the valve may selectively establish a desired fluid pathway through the manifold. A seal, which may include first and second sealing elements and a land extending between the first and second sealing elements, may be interposed between the actuatable valve and the housing. The first and second sealing elements may be configured to provide a fluid tight seal between the housing and the actuatable valve when the valve is in either the first or second position, and the land may be configured to provide a fluid tight seal between the housing and the actuatable valve when the valve is intermediate the first and second positions.

Yet another embodiment includes a manifold including a housing having a plurality of ports and a plurality of bores networking the plurality of ports, and an actuatable valve having one or more bores selectively in fluid communication with one or more of the plurality of bores of the housing. A seal may be interposed between the housing and the actuatable valve. The seal may include a primary seal portion configured to provide a fluid tight seal between the housing and the actuatable valve when one or more of the bores of the actuatable valve is aligned with one or more of the plurality of bores of the housing, and a secondary seal portion configured to provide a fluid tight seal between the housing and the actuatable valve when one or more of the bores of the actuatable valve is misaligned with one ore more of the plurality of bores of the housing.

### BRIEF DESCRIPTION OF THE DRAWINGS

The invention may be more completely understood in consideration of the following detailed description of various embodiments in connection with the accompanying drawings, in which:

FIG. 1A is a perspective view of an illustrative embodiment of an exemplary manifold;

FIG. 1B is an alternate perspective view of the exemplary manifold of FIG. 1A;

FIG. 2 is an exploded view of the exemplary manifold of FIGS. 1A and 1B;

FIG. 3A is a perspective top view of an illustrative embodiment of an exemplary sealing member for use in the exemplary manifold of FIGS. 1A and 1B;

FIG. 3B is a perspective bottom view of the exemplary sealing member of FIG. 3A;

FIG. 4 is plan view of the exemplary sealing member of FIGS. 3A and 3B;

FIG. 5 is a cross-sectional view of the exemplary sealing member taken along line 5-5 of FIG. 4;

FIG. 6 is a cross-sectional view of the exemplary sealing member taken along line 6-6 of FIG. 4;

FIG. 7A is a cross-sectional view of the exemplary manifold of FIGS. 1A and 1B with the actuatable valve in a first position;

FIG. 7B is a cross-sectional view of the exemplary manifold of FIGS. 1A and 1B with the actuatable valve in a second position;

FIGS. 8A-8C are cross-sectional views of an another exemplary manifold with an actuatable valve in a first position, a second position and a third position, respectively;

FIG. 9A is a perspective top view of another illustrative embodiment of an exemplary sealing member for use in the exemplary manifold of FIGS. 8A-8C;

FIG. 9B is a perspective bottom view of the exemplary sealing member of FIG. 9A;

FIGS. 10A-10B are cross-sectional views of another exemplary manifold with an actuatable valve in a first position and a second position, respectively;

FIG. 11A is a perspective top view of another illustrative embodiment of an exemplary sealing member for use in the exemplary manifold of FIGS. 10A and 10B; and

FIG. 11B is a perspective bottom view of the exemplary sealing member of FIG. 11A.

While the invention is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the invention.

### DETAILED DESCRIPTION

For the following defined terms, these definitions shall be applied, unless a different definition is given in the claims or elsewhere in this specification.

All numeric values are herein assumed to be modified by the term “about”, whether or not explicitly indicated. The term “about” generally refers to a range of numbers that one of skill in the art would consider equivalent to the recited value (i.e., having the same function or result). In many instances, the term “about” may be indicative as including numbers that are rounded to the nearest significant figure.

The recitation of numerical ranges by endpoints includes all numbers within that range (e.g., 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

The following detailed description should be read with reference to the drawings in which similar elements in different drawings are numbered the same. The detailed description and the drawings, which are not necessarily to scale, depict illustrative embodiments and are not intended to limit the scope of the invention. The illustrative embodiments depicted are intended only as exemplary. Selected features of any illustrative embodiment may be incorporated into an additional embodiment unless clearly stated to the contrary.

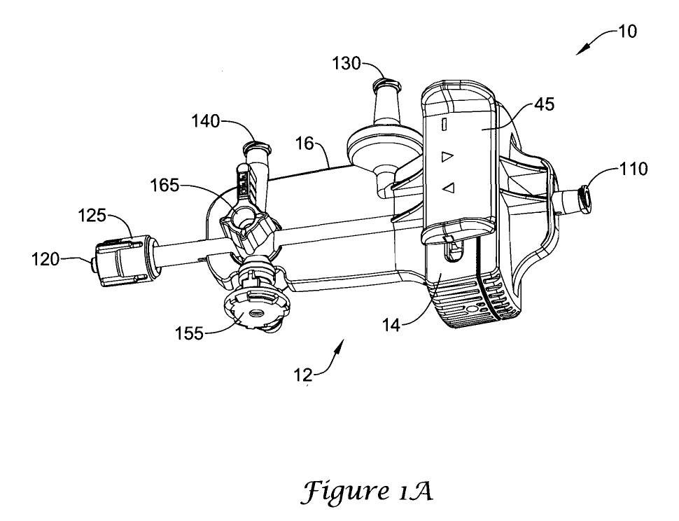

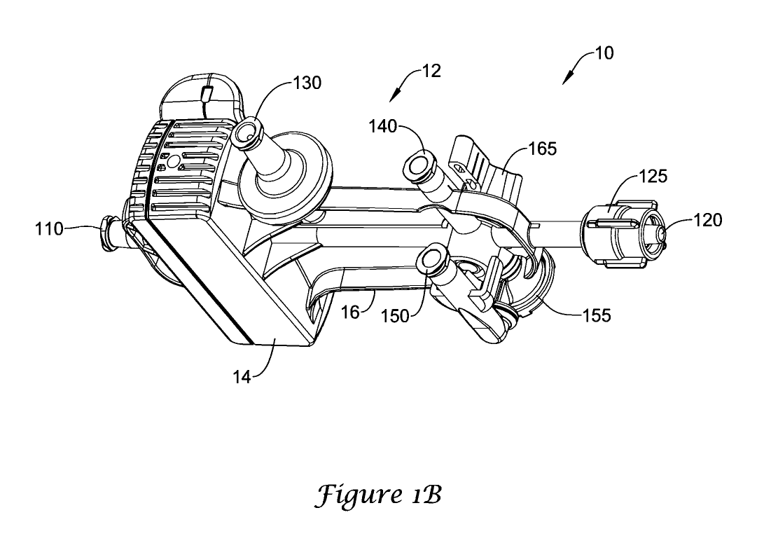

FIGS. 1A and 1B show perspective views of an exemplary manifold 10. The manifold 10 may simplify the control of multiple medical apparatuses and/or fluid pathways when coupled to a catheter or other medical device. The housing 12 of the manifold 10 may include multiple components. For example, the housing 12, which may include a valve housing 14 and a port housing 16, includes a plurality of ports extending from the housing 12. The ports may include ports for receiving medical apparatuses, such as therapeutic and/or diagnostic substance sources, a contrast media source, a saline source, a waste receptacle, pressure and/or suction devices, medical devices, monitoring devices, other fluid sources, receptacles, and/or lines, and the like. For example, a first port 110 may be configured to receive or be coupled to a pressure/suction device, such as a syringe. Thus, in some embodiments, the first port 110 may be characterized as a syringe port. A second port 120 may be configured to receive or be coupled to a catheter or other medical device. Thus, in some embodiments, the second port 120 may be characterized as a catheter port. Although characterized as a catheter port, it should be understood that the term catheter port is used in its most general sense as describing a port configured to be coupled to any single lumen or multi-lumen elongate medical device. The second port 120 is illustrated as including a rotating adaptor 125 allowing a catheter or other medical device coupled to the manifold 10 to be rotated independent of rotation of the manifold 10.

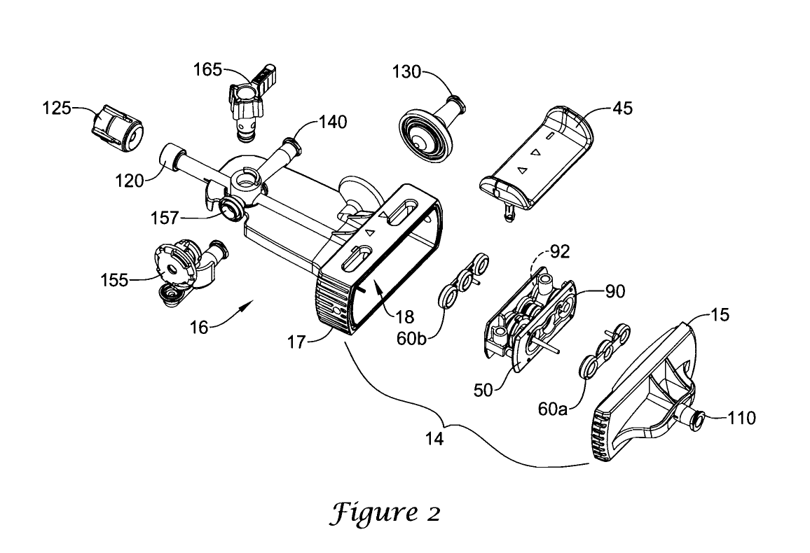

Additional ports of the illustrative embodiment may include a third port 130, which may be configured to be coupled to a contrast media source or other fluid source. Thus, in some embodiments the third port 130 may be characterized as a contrast port. A fourth port 140, which may be configured to be coupled to a saline/waste line and/or reservoir, may be characterized as a saline/waste port in some embodiments. A fifth port 150 is illustrated as a pressure monitoring port. The fifth port 150 may extend from a pressure transducer protection valve assembly 155 for monitoring and/or regulating pressure within the manifold 10. The pressure transducer protection valve assembly 155 may be attached to the housing 12 at the coupling point 157 (shown in FIG. 2).

Although the manifold 10 has been described as having five ports, the manifold 10 may include fewer or more ports as desired for a given medical application. For example, the manifold 10 may include two, three, four, five, six, or more ports as desired. Although certain ports have been described as associated with identified medical apparatuses, devices, and/or fluid sources, reservoirs and/or lines, it is contemplated that other apparatuses, devices, and/or fluid sources, reservoirs and/or lines may be coupled to the manifold 10 as well, either in addition to or as a replacement to those already noted.

The manifold 10 may additionally include one or more flow control valves, such as a stopcock valve 165. The stopcock valve 165 may be selectively rotated to manipulate the flow of fluids to/from one or more of the ports of the manifold 10. The stopcock valve 165 may be manually actuatable or automated. The functionality of the stopcock valve 165 will be described in greater detail with regard to the various fluid pathways of the manifold 10.

A wide variety of materials may be used in the construction of the manifold 10. Typical materials may include polymers, such as but not limited to, polycarbonate, acetal materials, acrylics, silicones, and polyolefins. Additional materials, such as natural materials, synthetic materials, as well as metallic materials may be used in the construction of the manifold 10 or a subset of its various components. Various components may be formed from a molding process, such as an injection molding process, a liquid injection molding process, a stamp molding process, a milling process, a compression molding process, casting, or other forming processes generally known in the art. The various components of the manifold 10 may be assembled using bonding techniques including solvents, adhesives such as UV cured adhesives; welding techniques including ultrasonic, thermal or spin techniques; or mechanical fits such as snap or interference fits, mechanical fasteners such as rivets, bolts, screws, clips, etc., and/or other mechanical attachment devices; as well as other means generally known in the art.

Additional components of the illustrative manifold 10 may be realized from the exploded view of FIG. 2. The manifold 10 may include an actuatable valve 50 configured to be actuated in order to selectively control fluid pathways through the manifold 10. For example, the actuatable valve 50 may be slidably actuated with an actuator such as an actuator button 45, or other actuation means not shown, such as one or more of an actuation lever, knob, handle, switch, spring, or the like. In some embodiments, the actuatable valve 50 may be actuated with an actuation force less than about 4 pounds, less than about 3.5 pounds, or less than about 3.25 pounds.

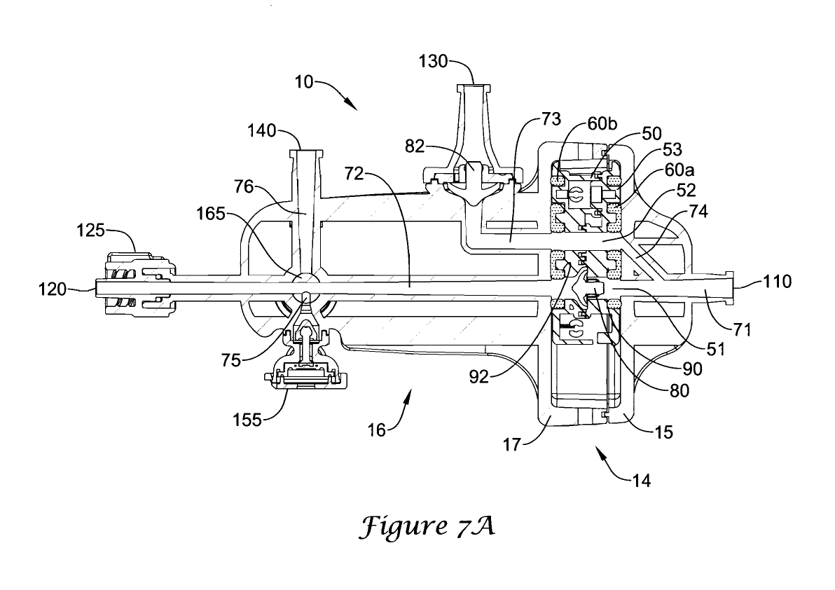

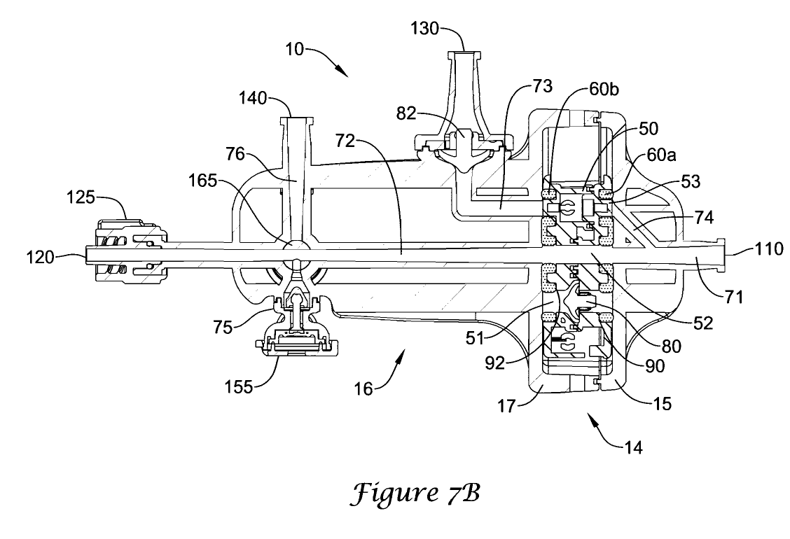

As shown in FIG. 2, the valve housing 14 may include multiple portions separable from one another, or the valve housing 14 may be a unitary component. In the illustrated embodiment, the valve housing 14 includes a first portion 15 and a second portion 17. The first portion 15 may include one or more, or a plurality of bores extending therein and may terminate at one or more, or a plurality of openings in the rear wall of the first portion 15. For example, the manifold 10 may include two bores (See FIGS. 7A and 7B) terminating at two openings in the rear wall of the first portion 15. In an assembled configuration, one side of the actuatable valve 50 may be adjacent to the rear wall of the first portion 15. The first portion 15 of the valve housing 14 may include one or more ports. As shown in FIG. 2, the first portion 15 may include the first port 110, which may be a pressure/suction device port for coupling a pressure/suction device such as a syringe to the manifold 10.

One or more of the first and second portions 15, 17 may include a recess or cavity for receiving the actuatable valve 50 or a portion thereof. For example, the second portion 17 may include a cavity 18 for receiving the actuatable valve 50. The second portion 17 may be adjacent the port housing 12 such that one or more, or a plurality of bores extending within the port housing 12 may terminate at one or more, or a plurality of openings in the rear wall of the second portion 17 within the cavity 18. For example, the manifold 10 may include two bores (See FIGS. 7A and 7B) terminating at two openings in the rear wall of the second portion 17. In an assembled configuration, one side of the actuatable valve 50 may be adjacent to the rear wall of the second portion 17.

The first portion 15 may be configured to mate with the second portion 17, such that the actuatable valve 50 may be enclosed in the cavity 18 of the valve housing 14. The first portion 15 and the second portion 17 may be assembled using bonding techniques including solvents, adhesives such as UV cured adhesives; welding techniques including ultrasonic, thermal or spin techniques; or mechanical fits such as snap or interference fits, mechanical fasteners such as rivets, bolts, screws, clips, etc., and/or other mechanical attachment devices; as well as other means generally known in the art.

The manifold 10 may additionally include one or more seals for providing a fluid tight seal between the actuatable valve 50 and the housing 12. For example, as shown in FIG. 2, the manifold 10 may include a first seal 60a and a second seal 60b. The first seal 60a and the second seal 60b may be positioned on opposite sides of the actuatable valve 50, such that when the manifold 10 is in an assembled configuration, the first seal 60a may be positioned between, or interposed between, the actuatable valve 50 and the rear wall of the first portion 15 of the valve housing 14. Additionally or alternatively, the second seal 60b may be positioned between, or interposed between, the actuatable valve 50 and the rear wall of the second portion 17 of the valve housing 14. Thus, in some embodiments, such as the embodiment illustrated in FIG. 2, the first and second seals 60a, 60b may be positioned in the interfaces between the actuatable valve 50 and the valve housing 14. In an assembled configuration, the seals 60a, 60b may be compressed between the actuatable valve 50 and the valve housing 14. In the illustrated embodiment, the first seal 60a and the second seal 60b are substantially the same, thus discussion of only one seal 60 follows. However, in some embodiments, the first seal 60a and the second seal 60b may be dissimilar, each possessing individual characteristics as may be desired for a specific application.

In the illustrated embodiment the actuatable valve 50 includes first and second recessed grooves or glands 90, 92 on opposite sides of the actuatable valve 50 for receiving and seating the seals 60a, 60b. In other embodiments, one or both of the first and second portions 15, 17 of the valve housing 14 may include recessed grooves or glands for receiving and seating the seals 60a, 60b.

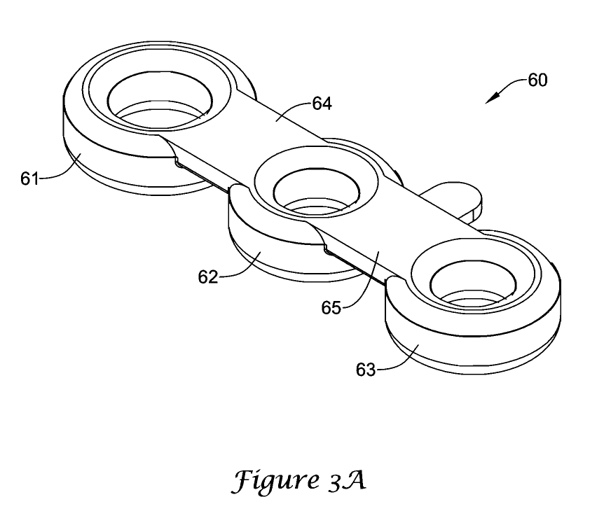

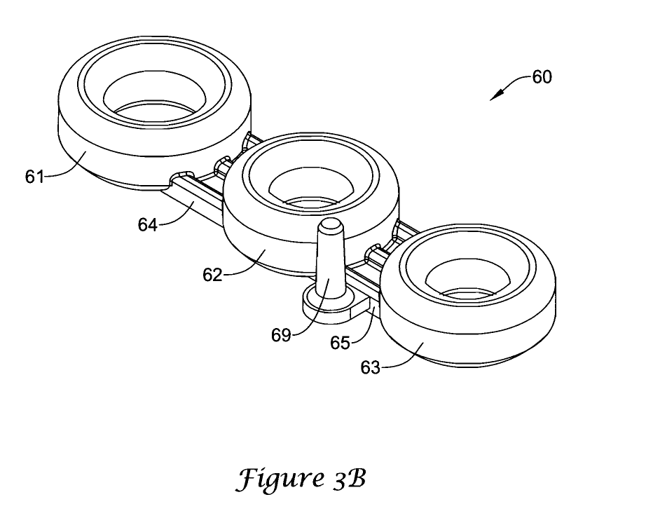

Referring to FIGS. 3A and 3B, the seal 60 may be further described. The seal 60 may include one or more, or a plurality of sealing elements. For example, the seal 60 may include one, or a plurality of primary sealing portions, such as a first sealing element 61, a second sealing element 62, and a third sealing element 63. Although the seal 60 is shown as including three sealing elements 61, 62, 63, in alternative embodiments the seal 60 may include one, two, four, or more sealing elements, such as annular sealing elements. Each of the sealing elements 61, 62, 63 may be any desired shape. For instance, each of the sealing elements 61, 62, 63 may be an annular sealing element resembling an O-ring type seal, a quad-ring seal, a lip seal, or a loaded lip seal in some embodiments. A secondary sealing portion, such as a land may connect each of the sealing elements 61, 62, 63 to an adjoining sealing element 61, 62, 63. For example, a first land 64 may extend between or bridge the first sealing element 61 with the second sealing element 62. Additionally, a second land 65 may extend between or bridge the second sealing element 62 with the third sealing element 63. In embodiments including additional sealing elements, additional lands may be included to connect or bridge the additional sealing elements with adjacent sealing elements. In the illustrative embodiment, the first, second, and third sealing elements 61, 62, 63 are aligned along a common axis. In other words, the center of each of the annuluses of the sealing elements 61, 62, 63 lies along a common axis. Likewise, each of the lands 64, 65 of the illustrative embodiment lies along and centered on the common axis of the seal 60. However, in other embodiments at least one of the sealing elements 61, 62, 63 may be misaligned with at least one other of the sealing elements 61, 62, 63 as desired, and/or at least one of the lands 64, 65 may be misaligned with one or more of the other components of the seal 60.

In some embodiments, the seal 60 may additionally include one or more, or a plurality of alignment members. For example, in the illustrative embodiment the seal 60 includes an alignment member depicted as a pin 69. The pin 69 may be incorporated in the seal 60 to provide alignment, positioning, and/or retention capabilities to the seal 60. For example, when assembled, the pin 69 may be inserted into a hole of another component of the manifold 10, such as the actuatable valve 50 and be retained therein.

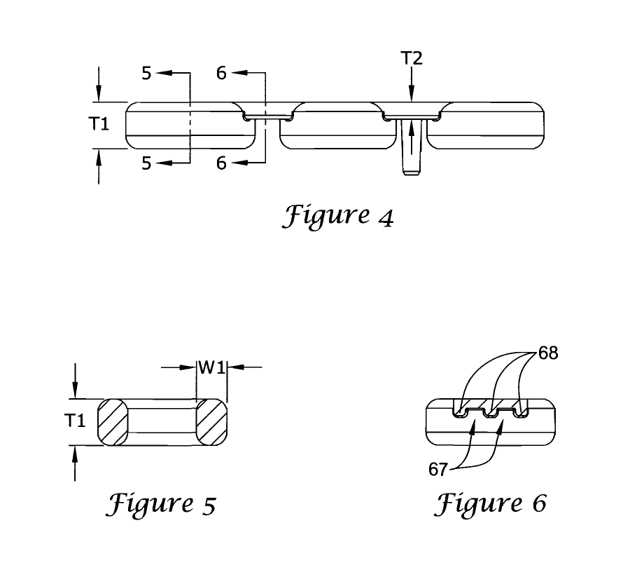

As shown in the plan view of FIG. 4, each of the sealing elements 61, 62, 63 may have a thickness, T₁. The thickness, T₁, is intended to describe the distance from one side of the sealing element 61, 62, 63 to the opposite side of the sealing element 61, 62, 63 in a direction parallel to the axial centerline of the opening of the sealing element 61, 62, 63, such as the axial centerline of the central opening of each of the annuluses of the sealing elements 61, 62, 63. Each of the lands 64, 65 may have a thickness, T₂. The thickness, T₂, of the lands 64, 65 is intended to describe the distance in the direction parallel to the thickness, T₁, of the sealing elements 61, 62, 63. In the illustrated embodiment, the thickness, T₁, of the sealing elements 61, 62, 63 is greater than the thickness, T₂, of the lands 64, 65. In some embodiments, the thickness, T₂, of the lands 64, 65 may be about 75%, about 50%, or about 25% of the thickness, T₁ of the sealing elements 61, 62, 63. However, in other embodiments, the relative thicknesses T₁ and T₂ may be of other proportions. Accordingly, sufficient backup support on the sealing elements 61, 62, 63 may be maintained while being subjected to operative fluid pressures. A larger ratio of T₁ to T₂ (i.e., the higher the T₁/T₂ ratio) leads to more backup support and/or higher pressure capability of the seal. In other embodiments, the thickness, T₁, may be equal to, or substantially equal to the thickness, T₂. In still other embodiments, the thickness, T₂, may be greater than the thickness T₁. As shown in FIG. 4, one side (the top side) of the lands 64, 65 may be a smooth surface and/or may be flush with one side (the top side) of the sealing elements 61, 62, 63. However, in other embodiments, the lands 64, 65 may be positioned other than flush with the sealing elements 61, 62, 63.

FIG. 5 is a cross-sectional view of the first sealing element 61 taken along line 5-5 of FIG. 4. In the illustrative embodiment, the sealing element 61 may be representative of each of the sealing elements 61, 62, 63. However, in other embodiments, the first sealing element 61, second sealing element 62, and/or the third sealing element 63 may be dissimilar to one or more of the other sealing elements 61, 62, 63 if desired. As shown in FIG. 5, the sealing element 61 may have an elliptical or oval cross-section. However, in other embodiments, the cross-section of the sealing elements 61, 62, 63 may be circular, non-circular, polygonal, trapezoidal, hexagonal, or any other desired shape. In embodiments where the sealing elements 61, 62, 63 include non-circular or non-regular polygonal cross-sections, the thickness, T₁, of the sealing elements 61, 62, 63 may be chosen to be greater than the width, W₁, of the sealing elements 61, 62, 63. The width, W₁, is intended to describe the distance in the direction perpendicular to the thickness, T₁, across the cross-section of the sealing elements 61, 62, 63. For example, in embodiments wherein the sealing elements 61, 62, 63 are annular sealing elements, the width, W₁, is determined as one half of the difference between the outer diameter and the inner diameter of the annular sealing element. Thus, in embodiments wherein the thickness, T₁, is greater than the width, W₁, the sealing elements 61, 62, 63 may have a greater dimension in the direction of compression than in the transverse direction when the sealing elements 61, 62, 63 are assembled in the valve housing 14. Among other things, this may alleviate the precision of dimensional tolerances of components of the manifold 10 without compromising the sealing capabilities of the manifold 10.

FIG. 6 is a cross-sectional view of the first land 64 taken along line 6-6 of FIG. 4. In the illustrated embodiment, the land 64 may be representative of each of the lands 64, 65. However, in other embodiments, the first land 64 may be dissimilar to the second land 65, if desired. The land 64 may be described as a band of material extending between adjacent sealing elements. The land 64 may include one or more, or a plurality of voids 67. The void(s) 67 may reduce the amount of material necessary to form the land 64. For example, the void(s) 67 may define two or more, or a plurality of ribs 68 along the land 64. Thus, the ribs 68 may provide a raised portion of the land 64. The ribs 68 may reduce the frictional resistance between the lands 64, 65 and an adjacent component. Alternatively, a raised portion of the lands 64, 65 may include projections, such as a plurality of bumps, knobs, convex surfaces and/or protuberances. In other embodiments, the lands 64, 65 may include a honeycomb webbing or other band of material including a plurality of voids and/or raised portions. Thus, when the lands 64, 65 are compressed between the actuatable valve 50 and valve housing 14, less than the entire surface area of the bottom surface of the lands 64, 65 contacts the adjacent surface of the valve housing 14 and/or the actuatable valve 50, yet maintaining sufficient contact pressure between the sealing surface of the seal 60 and the housing 12. This arrangement may reduce the amount of actuation force necessary to actuate the actuatable valve 50 within the valve housing 14.

The seal 60 may comprise a material having notable material properties. Some suitable materials may be elastomeric, resilient, inert, biocompatible, compressible and/or incompressible. One suitable material for the seal 60 may be silicone. Other suitable materials for the seal 60 include, but are not limited to, polytetrafluoroethylene (PTFE), polyurethane, neoprene, ethylene propylene, and additional fluoropolymer elastomers.

Referring to FIGS. 7A and 7B, select internal features of the manifold 10 may be further described. The manifold 10 may include a plurality of bores networking the plurality of ports. For example, a first bore 71 may be in fluid communication with the first port 110, a second bore 72 may be in fluid communication with the second port 120, a third bore 73 may be in fluid communication with the third port 130, a fourth bore 74 may be in fluid communication with and branch from the first bore 71, a fifth bore 75 may be in fluid communication with the fifth port 150 (See FIGS. 1A and 1B), and/or a sixth bore 76 may be in fluid communication with the fourth port 140. Each of the first, second, third and fourth bores 71, 72, 73, 74 may terminate at an opening in the housing 12 juxtaposed to the valve 50. In alternative embodiments, additional bores may extend through the manifold 10 to provide additional and/or alternative fluid pathways through the manifold 10 to/from one of a plurality of ports.

The actuatable valve 50 may include one, or a plurality of bores extending therethrough. For example, in the illustrative example, the actuatable valve 50 includes a first bore 51 extending from a first side of the actuatable valve 50 to an opposing side of the valve 50. A second bore 52 may also extend from a first side of the actuatable valve 50 to the opposite side of the valve 50. The actuatable valve 50 may additionally include a blind bore or dummy position 53 not establishing a fluid pathway through the valve 50.

One or more of the bores through the actuatable valve 50 may include a check valve, such as a one-way or a two-way check valve. For example, the first bore 51 may include a check valve 80, illustrated as a one-way check valve, for restricting fluid flow therethrough. In other embodiments, the second bore 52 may include a check valve in addition to or instead of the check valve 80 in the first bore 51.

Additional check valves may be positioned at additional locations of the manifold 10 as desired to provide a desired controllability of fluid flow through the manifold 10. For example, a second check valve 82, illustrated as a one-way check valve, may be positioned in the fluid flow path defined by the third port 130 and the third bore 73. Although the second check valve 82 is shown proximate the third port 130, the second check valve 82 may be positioned at any desired position along the defined fluid flow path. For example, in an alternative embodiment, the second check valve 82 may be positioned in the second bore 52 of the actuatable valve 50 or the second check valve 82 may be positioned external of the manifold 10.

Check valves, such as the check valves 80, 82, may be accomplished by any suitable structure or technique, including but not limited to umbrella valves, duckbill valves, disk valves, ball check valves, flapper type valves, and the like. Additionally, different types of valves may be incorporated at different locations within the same embodiment.

The stopcock 165, which may be positioned in the fluid pathway of one or more of the bores 71, 72, 73, 74, 75, 76, may be used to selectively control and/or regulate fluid passing through the manifold 10. For example, in the illustrative embodiment, the stopcock 165 may be positioned in the fluid pathway of the second bore 72, the fifth bore 75 and the sixth bore 76. The stopcock 165 may be a three-way stopcock, thus rotating the stopcock 165 between one of three positions may alter the fluid pathway through one or more of the second, fifth, and sixth bores 72, 75, 76.

The functionality of the manifold 10 may now be described while referring to FIGS. 7A and 7B, which illustrate two operational configurations of the manifold 10. The actuatable valve 50 may be actuated between one of multiple positions. For example, in the illustrative embodiment, the actuatable valve 50 may be actuated between two positions. For instance, the actuatable valve 50 may be actuated by sliding the actuatable valve 50 between a first position and a second position. However, in other embodiments, the actuatable valve 50 may be configured to be actuated between three, four, five or more positions as desired.

FIG. 7A shows the actuatable valve 50 in a first position. With the actuatable valve 50 in the first position, the second bore 52 of the actuatable valve 50 may be aligned with the openings of the third bore 73 and the fourth bore 74 of the housing 12. Thus, the third port 130, which may be characterized as the contrast port in some embodiments, may be in fluid communication with the first port 110, which may be coupled to a pressure/suction device, such as a syringe. In the first position, suction may be applied at the first port 110, thus drawing fluid from a fluid source coupled to the third port 130 through a fluid pathway defined in the manifold 10 to the pressure/suction device coupled to the first port 110. The fluid pathway may be characterized as the pathway from the third port 130, through the check valve 82, through the third bore 73, through the second bore 52 of the actuatable valve 50, through the fourth bore 74, through the first bore 71, to the first port 110. Thus, in the illustrative embodiment, the first position may be characterized as a contrast fill position, as contrast media coupled to the third port 130 may be drawn into a pressure/suction device coupled to the first port 110 while in the first position. If the check valves 80, 82 are included in the embodiment, as suction is applied from the first port 110 by a pressure/suction device, the first check valve 80 prevents retrograde flow from the second bore 72 from entering the first bore 71, while the second check valve 82 allows fluid flow therethrough.

Additionally, with the actuatable valve 50 in the first position, the first bore 51 of the actuatable valve 50 may be aligned with the openings of the first bore 71 and the second bore 72 of the housing 12. Thus, the first port 110 may be in fluid communication with the second port 120, which may be a catheter port. Pressure may be applied at the first port 110 with the pressure/suction device or another device, thus thrusting a fluid through a fluid pathway defined in the manifold 10 to a catheter or other medical device coupled to the second port 120. The fluid pathway may be characterized as the pathway from the first port 110, through the first bore 71, through the first check valve 81 in the first bore 51 of the actuatable valve 50, through the second bore 72, to the second port 120. Thus, in the illustrative embodiment, the first position may additionally or alternatively be characterized as a contrast fill position. If the check valves 80, 82 are included in the embodiment, as pressure is applied from the first port 110 by a pressure/suction device, the first check valve 80 allows fluid flow from the first bore 71 through the actuatable valve 50 to the second bore 72 and to the second port 120, while the second check valve 82 prevents retrograde fluid flow through the third bore 73 and exiting the third port 130.

Thus, while in the first position, a fluid, such as a contrast media, may be drawn into a pressure/suction device coupled to the first port 110. Once a desired quantity of fluid has been drawn into the pressure/suction device, positive pressure may be applied at the first port 110 by the pressure/suction device in order to expel the drawn fluid out of the second port 120 and into a catheter or other medical device during a medical procedure.

While in the first position, the primary sealing portions, illustrated as annular sealing members of the seal 60, provide a fluid tight seal between the actuatable valve 50 and the valve housing 14 to prevent fluid leakage or bypass through the interface between the actuatable valve 50 and the valve housing 14 of the manifold 10. In some embodiments, the primary seal portion (e.g. the annular sealing members) may withstand pressures of 300 psi or greater, 400 psi or greater, or 500 psi or greater. The seal 60 may be placed or seated in a gland 90, 92 of the actuatable valve 50 to correctly position and/or prevent backup of the seal 60 under operative fluid pressures. As shown in the illustrated embodiment, the annuluses of the primary sealing portions of the seal 60 may be centered on the openings of the first, second, and third bores 51, 52, 53 of the actuatable valve 50.

The actuatable valve 50 may be actuated between the first position and the second position. For example, by applying a force to the actuation button 45, the valve 50 may be slidably actuated between the first position and the second position. In some embodiments, the valve 50 may be actuated between the first position and the second position with an actuation force less than about 4 pounds, less than about 3.5 pounds, or less than about 3.25 pounds. While the actuatable valve 50 is at a transitional position intermediate the first position and the second position, the secondary sealing portion of the seal 60, illustrated as the lands 64, 65, may provide a fluid tight seal between the valve 50 and the valve housing 14 to prevent fluid leakage or bypass through the interface between the actuatable valve 50 and the valve housing 14 of the manifold 10 during actuation of the valve 50, since while in a transitional position intermediate the first position and the second position, one or more of the bores of the valve 50 may be misaligned with one or more of the bores of the housing 14.

FIG. 7B shows the actuatable valve 50 in the second position. With the actuatable valve 50 in the second position, the second bore 52 of the valve 50 may be aligned with the openings of the first bore 71 and the second bore 72 of the housing 12. Thus, the second port 120, which may be characterized as the catheter port in some embodiments, may be in fluid communication with the first port 110, which may be coupled to a pressure/suction device, such as a syringe. Additionally, with the actuatable valve 50 in the second position, the blind bore or dummy position 53 of the actuatable valve 50 may be aligned with the openings of the third bore 73 and the fourth bore 74 of the housing 12. In this position, the blind bore or dummy position 53 prevents fluid flow between the third bore 73 and the fourth bore 74. In the second position, a pressure/suction device coupled to the first port 110 may be used to draw fluid out of or aspirate fluid from the manifold 10 and/or a catheter or other medical device coupled to the second bore 120. Such aspiration may be used, for example, to clear the catheter and/or the manifold 10 of any air or coagulated blood either after a catheter exchange takes place, prior to catheter insertion, or after a period of non-use in a medical procedure. For instance, it is often considered appropriate to aspirate the catheter and/or manifold 10 after an idle period, such as two minutes, where no fluid is infused or aspirated during a procedure in order to clear any air bubbles or coagulated blood that may form during such time. Additionally or alternatively, fluid may be infused into the medical device coupled to the second bore 120 while the actuatable valve 50 is in the second position.

In embodiments wherein aspiration of fluid is performed, fluid aspirated from the catheter can be cleared from the pressure/suction device by rotating the stopcock 165, for example 90° clockwise or counter-clockwise. Rotation of the stopcock 165 selectively closes the pathway to the second port 120 and opens one or both of ports 140, 150. For example, rotation of the stopcock 165 may open a pathway to the fourth port 140, which may be coupled to a saline/waste line and/or reservoir. Thus, the fluid pathway may be characterized as the pathway from the first port 110, through the first bore 71, through the second bore 52 of the actuatable valve 50, through the second bore 72, through the sixth bore 76, to the fourth port 140. Applying pressure to the pressure/suction device coupled to the first port 110 may initiate fluid flow through the sixth bore 76 and out the fourth port 140 of the housing 12.

Rotation of the stopcock 165 to another position may open a pathway to the fifth port 150, via the pressure transducer protection valve assembly 155. Thus, the fluid pathway may be characterized as the pathway from the first port, 110, through the first bore 71, through the second bore 52 of the actuatable valve 50, through the second bore 72, through the fifth bore 75 to the fifth port 150. In this position, the pressure transducer protection valve assembly 155, in communication with the fluid flow pathway, may monitor and/or regulate the pressure at the fifth port 150. In other embodiments, which may or may not include a pressure transducer protection valve assembly 155, the fifth port 150 may be utilized for auxiliary functions.

Thus, while in the second position, fluid may be aspirated from a medical device coupled to the manifold 10 by drawing the fluid into a pressure/suction device coupled to the first port 110. Once a desired quantity of fluid has been drawn into the pressure/suction device, the stopcock 165 may be rotated to open an alternate fluid pathway to the fourth port 140. Then, positive pressure may be applied at the first port 110 by the pressure/suction device in order to expel the drawn fluid out of the fourth port 140 and into a waste reservoir during a medical procedure. Alternatively, fluid from the fourth port 140 may be drawn into the pressure/suction device coupled to the first port 110. Once a desired quantity of fluid has been drawn into the pressure/suction device, the stopcock 165 may be rotated to open an alternate fluid pathway to the second port 120. Then, positive pressure may be applied at the first port 110 by the pressure/suction device in order to expel the drawn fluid out of the second port 120 and into the catheter or other medical device coupled to the second port 120 during a medical procedure.

While in the second position, the primary sealing portions, illustrated as annular sealing members of the seal 60, provide a fluid tight seal between the actuatable valve 50 and the valve housing 14 to prevent fluid leakage or bypass through the interface between the actuatable valve 50 and the valve housing 14 of the manifold 10. In some embodiments, the primary seal portion (e.g. the annular sealing members) may withstand pressures of 300 psi or greater, 400 psi or greater, or 500 psi or greater. The seal 60 may be placed or seated in a gland 90, 92 of the actuatable valve 50 to correctly position and/or prevent backup of the seal 60 under operative fluid pressures.

The fluid pathways described above which may be established through the manifold 10 are intended as exemplary. Those skilled in the art will recognize that additional fluid pathways through the manifold 10 may be established for a given medical procedure by selectively actuating the valve 50 between the first and second positions and/or rotating the stopcock 165 between one of several positions, or by including additional flow control devices in the manifold 10.

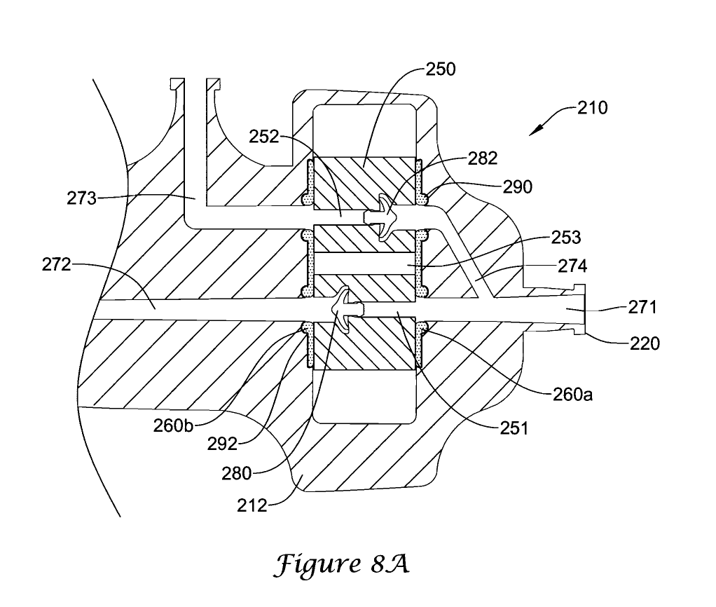

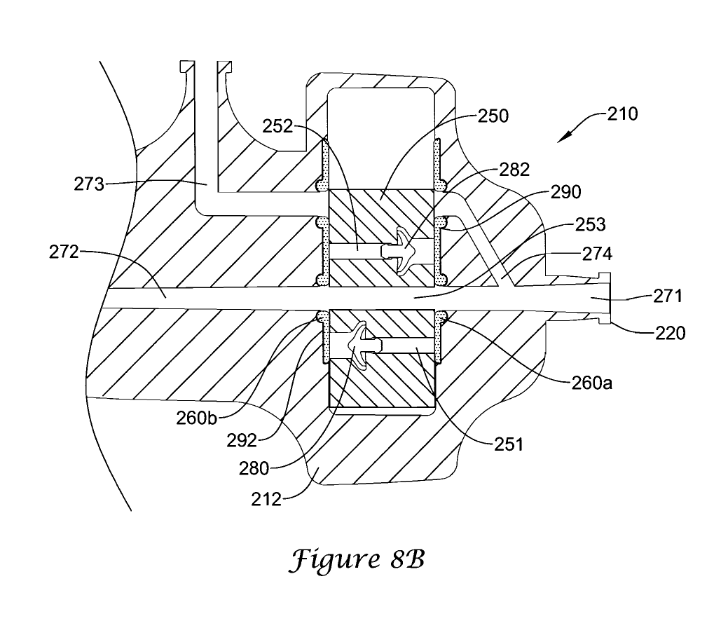

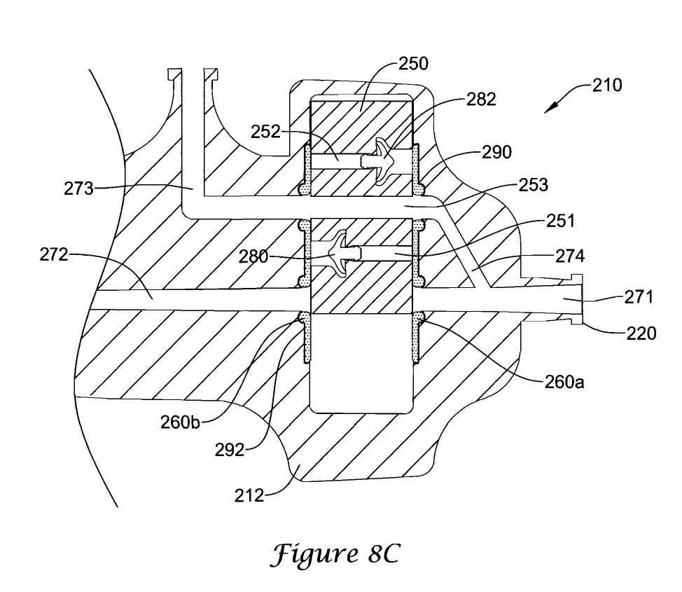

FIGS. 8A-8C illustrate an alternate embodiment of a manifold 210 including a housing 212 and an actuatable valve 250. In the illustrative embodiment, the valve 250 may include a plurality of bores, such as a first bore 251, a second bore 252, and a third bore 253. The valve 250 may include one or a plurality of check valves, such as one-way or two-way check valves, for controlling fluid flow through the valve 250. As illustrated in FIGS. 8A-8C, the valve 250 may include a first check valve 280, illustrated as a one-way check valve, positioned in the first bore 251, and a second check valve 282, illustrated as a one-way check valve, positioned in the second bore 252. The first check valve 280 may be positioned such that the first check valve 280 provides uni-directional fluid flow in a first direction, and the second check valve 282 may be positioned such that the second check valve 282 provides uni-directional fluid flow in a second direction, opposite the uni-directional fluid flow through the first check valve 280. As illustrated in FIGS. 8A-8C, the third bore 253 may be configured to allow multi-directional fluid flow through the valve 250.

The housing 212 includes a plurality of bores, including a first bore 271, a second bore 272, a third bore 273, and a fourth bore 274, extending therein. Each of the bores 271, 272, 273, 274 may terminate at an opening in the housing 212 juxtaposed to the valve 250.

Additionally, the manifold 210 may include one or more seals for providing a fluid tight seal between the actuatable valve 250 and the housing 212. For example, the manifold 210 may include a first seal 260a and a second seal 260b. The first seal 260a and the second seal 260b may be positioned on opposite sides of the actuatable valve 250, such that when the manifold 210 is in an assembled configuration, the first seal 260a may be positioned between, or interposed between, the actuatable valve 250 and the housing 212. Additionally or alternatively, the second seal 260b may be positioned between, or interposed between, the actuatable valve 250 and the housing 212. Thus, in some embodiments, such as the embodiment illustrated in FIGS. 8A-8C, the first and second seals 260a, 260b may be positioned in the interfaces between the actuatable valve 250 and the housing 212. In an assembled configuration, the seals 260a, 260b may be compressed between the actuatable valve 250 and the housing 212. In the illustrated embodiment, the first seal 260a and the second seal 260b are substantially the same, thus discussion of only one seal 260 follows. However, in some embodiments, the first seal 260a and the second seal 260b may be dissimilar, each possessing individual characteristics as may be desired for a specific application.

In the illustrated embodiment, the housing 212 includes first and second recessed grooves or glands 290, 292 on opposite sides of the actuatable valve 250 for receiving and seating the seals 260a, 260b. In other embodiments, however, the actuatable valve 250 may include recessed grooves or glands for receiving and seating the seals 260a, 260b.

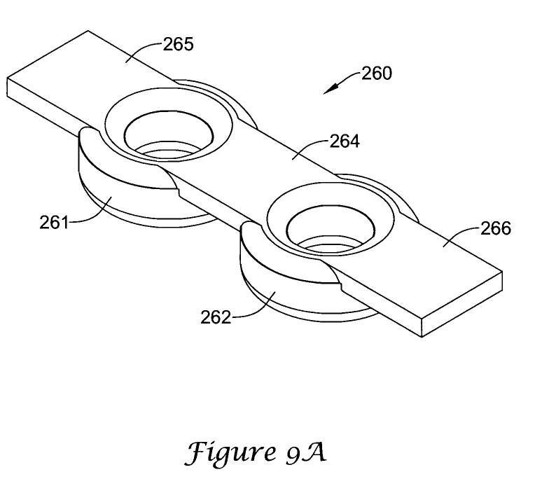

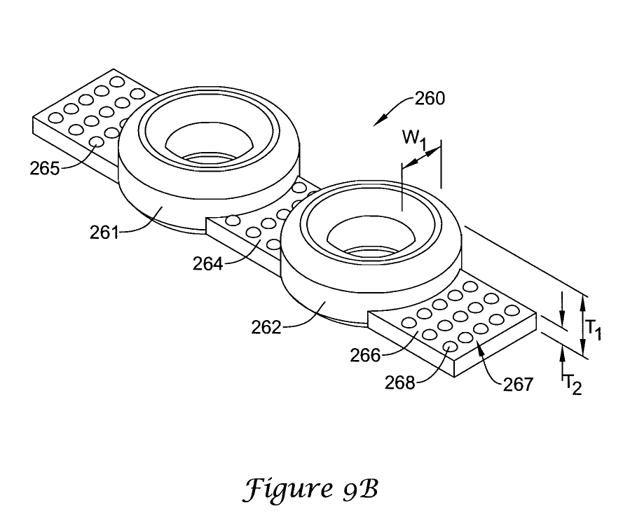

Referring to FIGS. 9A and 9B, the seal 260 may be further described. Similar to the seal 60, the seal 260 may include one or more, or a plurality of sealing elements. For example, the seal 260 may include one, or a plurality of primary sealing portions, such as a first sealing element 261 and a second sealing element 262. Although the seal 260 is shown as including two sealing elements 261, 262, in alternative embodiments the seal 260 may include one, three, four or more sealing elements, such as annular sealing elements. Each of the sealing elements 261, 262 may be any desired shape. For instance, each of the sealing elements 261, 262 may be an annular sealing element resembling an O-ring type seal. A secondary sealing portion, such as a land 264 may connect each of the sealing elements 261, 262 to the adjoining sealing element 261, 262. For example, the land 264 may extend between or bridge the first sealing element 261 with the second sealing element 262. In embodiments including additional sealing elements, additional lands may be included to connect or bridge the additional sealing elements with adjacent sealing elements. Additional secondary sealing portions of the seal 260, illustrated as a second land 265 and a third land 266, may extend from the primary sealing portions of the seal 260. For example, the second land 265 may extend from the first sealing element 261 and the third land 266 may extend from the second sealing element 262. Each of the second and third lands 265, 266 may resemble the land 264, except each of the second and third lands 265, 266 are only connected to one of the sealing elements 261, 262. In the illustrative embodiment, the first and second sealing elements 261, 262 are aligned along a common axis. In other words, the center of each of the annuluses of the sealing elements 261, 262 lies along a common axis. Likewise, each of the lands 264, 265, 266 of the illustrative embodiment lies along and is centered on the common axis of the seal 260. However, in other embodiments at least one of the sealing elements 261, 262 and/or at least one of the lands 264, 265, 266 may be misaligned with one or more of the other components of the seal 260.

Similar to the sealing elements 61, 62, 63 of the seal 60, each of the sealing elements 261, 262 may have a thickness, T₁. The thickness, T₁, is intended to describe the distance from one side of the sealing element 261, 262 to the opposite side of the sealing element 261, 262 in a direction parallel to the axial centerline of the opening of the sealing element 261, 262, such as the axial centerline of the central opening of each of the annuluses of the sealing elements 261, 262. Additionally, each of the lands 264, 265, 266 may have a thickness, T₂. The thickness, T₂, of the lands 264, 265, 266 is intended to describe the distance in the direction parallel to the thickness, T₁, of the sealing elements 261, 262. In the illustrated embodiment, the thickness, T₁, of the sealing elements 261, 262 is greater than the thickness, T₂, of the lands 264, 265, 266. In some embodiments, the thickness, T₂, of the lands 264, 265, 266 may be about 75%, about 50%, or about 25% of the thickness, T₁ of the sealing elements 261, 262. However, in other embodiments, the relative thicknesses T₁ and T₂ may be of other proportions. Accordingly, sufficient backup support on the sealing elements 261, 262 may be maintained while being subjected to operative fluid pressures. A larger ratio of T₁ to T₂ (i.e., the higher the T₁/T₂ ratio) leads to more backup support and/or higher pressure capability of the seal. In other embodiments, the thickness, T₁, may be equal to, or substantially equal to the thickness, T₂. In still other embodiments, the thickness, T₂, may be greater than the thickness T₁. As shown in FIG. 9A, one side (the top side) of the lands 264, 265, 266 may be a smooth surface and/or may be flush with one side (the top side) of the sealing elements 261, 262. However, in other embodiments, the lands 264, 265, 266 may be positioned other than flush with the sealing elements 261, 262.

In the illustrative embodiment, the sealing elements 261, 262 may be substantially similar. However, in other embodiments, the first sealing element 261 and the second sealing element 262 may be dissimilar if desired. Similar to the sealing elements 61, 62, 63 of the seal 60, the sealing elements 261, 262 may have an elliptical or oval cross-section. However, in other embodiments, the cross-section of the sealing elements 261, 262 may be circular, non-circular, polygonal, trapezoidal, hexagonal, or any other desired shape. In embodiments where the sealing elements 261, 262 include non-circular or non-regular polygonal cross-sections, the thickness, T₁, of the sealing elements 261, 262 may be chosen to be greater than the width, W₁, of the sealing elements 261, 262. The width, W₁, is intended to describe the distance in the direction perpendicular to the thickness, T₁, across the cross-section of the sealing elements 261, 262. For example, in embodiments wherein the sealing elements 261, 262 are annular sealing elements, the width, W₁, is determined as one half of the difference between the outer diameter and the inner diameter of the annular sealing element. Thus, in embodiments wherein the thickness, T₁, is greater than the width, W₁, the sealing elements 261, 262 may have a greater dimension in the direction of compression than in the transverse direction when the sealing elements 261, 262 are assembled in the housing 212. Among other things, this may alleviate the precision of dimensional tolerances of components of the manifold 210 without compromising the sealing capabilities of the manifold 210.

Each of the lands 264, 265, 266 may be described as a band of material extending from the sealing elements 261, 262. The land 264 may extend between or bridge the sealing elements 261, 262, and the lands 265, 266 may extend from one of the sealing elements 261, 262. The lands 264, 265, 266 may include a plurality of raised portions 268 defining voids 267 therebetween. The voids 267 may reduce the amount of material necessary to form the lands 264, 265, 266. For example, the raised portions 268 may define two or more, or a plurality of protrusions or bumps extending outward from the lands 264, 265, 266. Thus, the raised portions 268 may provide contact surfaces of the lands 264, 265, 266. The raised portions 268 may reduce the frictional resistance between the lands 264, 265, 266 and an adjacent component. Alternatively, the raised portions 268 of the lands 264, 265, 266 may include ribs, convex surfaces and/or other protuberances extending outward from the lands 264, 265, 266. In other embodiments, the lands 264, 265, 266 may include a honeycomb webbing or other band of material including a plurality of voids and/or raised portions. Thus, when the lands 264, 265, 266 are compressed between the actuatable valve 250 and the housing 212, less than the entire surface area of the bottom surface of each of the lands 264, 265, 266 contacts the adjacent surface of the housing 212 and/or the actuatable valve 250, yet maintaining sufficient contact pressure between the sealing surface of the seal 260 and the housing 212. This arrangement may reduce the amount of actuation force necessary to actuate the actuatable valve 250 within the housing 212.

The seal 260 may comprise a material having notable material properties. Some suitable materials may be elastomeric, resilient, inert, biocompatible, compressible and/or incompressible. One suitable material for the seal 260 may be silicone. Other suitable materials for the seal 260 include, but are not limited to, polytetrafluoroethylene (PTFE), polyurethane, neoprene, ethylene propylene, and additional fluoropolymer elastomers.

The functionality of the manifold 210 may now be described while referring to FIGS. 8A-8C, which illustrate three operational configurations of the manifold 210. The actuatable valve 250 may be actuated between one of multiple positions. For example, in the illustrative embodiment, the valve 250 may be actuated between three positions. For instance, the valve 250 may be actuated by sliding the valve 250 between a first position, a second position and/or a third position. In some embodiments, the valve 250 may be actuated between the first, second and third positions with an actuation force less than about 4 pounds, less than about 3.5 pounds, or less than about 3.25 pounds.

In the first position, shown in FIG. 8A, the first bore 251 of the actuatable valve 250 may be aligned with the openings of the first bore 271 and the second bore 272 of the housing 212. Thus, a fluid pathway may be defined through the housing 212 from the first bore 251, through the first check valve 280 positioned in the first bore 251 of the actuatable valve 250, and through the second bore 272. In this position, the inclusion of the first check valve 280 may allow uni-directional fluid flow from the first bore 271 to the second bore 272, but may prevent retrograde flow from the second bore 272 to the first bore 271. Additionally, in the first position, the second bore 252 of the actuatable valve 250 may be aligned with the openings of the third bore 273 and the fourth bore 274 of the housing 212. Thus, a fluid pathway may be defined through the housing 212 from the third bore 273, through the second check valve 282 positioned in the second bore 252 of the actuatable valve 250, and through the fourth bore 274. In this position, the inclusion of the second check valve 282 may allow uni-directional fluid flow from the third bore 273 to the fourth bore 274, but may prevent retrograde flow from the fourth bore 274 to the third bore 273.

In the first position, a pressure/suction device, such as a syringe, may be coupled to the port 220 and be in fluid communication with the first bore 271. Suction may be applied at the port 220 by the pressure/suction device, thus drawing fluid from a medical apparatus, such as therapeutic and/or diagnostic substance sources, a contrast media source, a saline source, a waste receptacle, monitoring devices, other fluid sources, receptacles, and/or lines, and the like, in fluid communication with the third bore 273 through the fluid pathway to the pressure/suction device. The inclusion of the first check valve 280 prevents retrograde fluid flow from the second bore 272 from entering the first bore 271, while the second check valve 282 allows fluid flow from the third bore 273 to the fourth bore 274.

Additionally or alternatively, in the first position, pressure from a pressure/suction device, such as a syringe, coupled to the port 220 may expel fluid from the pressure/suction device through the first bore 271 and second bore 272 to a catheter, or other medical device, in fluid communication with the second bore 272. The inclusion of the second check valve 282 prevents retrograde fluid flow from the fourth bore 274 from entering the third bore 273, while the first check valve 280 allows fluid flow from the first bore 271 to the second bore 272.

In the second position, shown in FIG. 8B, the third bore 253 of the actuatable valve 250 may be aligned with the openings of the first bore 271 and the second bore 272 of the housing 212. In this position, the third bore 253 of the actuatable valve 250 may allow multi-directional fluid flow through the valve 250 between the first bore 271 and the second bore 272. Additionally, in the second position, the actuatable valve 250 may interrupt the fluid flow pathway between the third bore 273 and the fourth bore 274, thus preventing fluid from passing between the third bore 273 and the fourth bore 274. Thus, in the second position, fluid may freely flow through the fluid pathway defined between the first bore 271, the third bore 253 of the valve 250, and the second bore 272 in the manifold 10 as pressure and/or suction is applied by a pressure/suction device coupled to the port 220.

In the second position, the secondary sealing portion, illustrated as the land 265, of the seals 260a, 260b, extends over the openings of the first bore 251 of the actuatable valve 250, maintaining a fluid tight seal between the first bore 251 of the actuatable valve 250 and the valve housing 212, thus preventing fluid flow through the first bore 251 and/or fluid bypass between the actuatable valve 250 and the valve housing 212.

In the third position, shown in FIG. 8C, the third bore 253 of the actuatable valve 250 may be aligned with the openings of the third bore 273 and the fourth bore 274 of the housing 212. In this position, the third bore 253 of the actuatable valve 250 may allow multi-directional fluid flow through the valve 250 between the third bore 273 and the fourth bore 274. Additionally, in the third position, the actuatable valve 250 may interrupt the fluid flow pathway between the first bore 271 and the second bore 272, thus preventing fluid from passing between the first bore 271 and the second bore 272. Thus, in the third position, fluid may freely flow through the fluid pathway defined between the third bore 273, the third bore 253 of the valve 250, the fourth bore 274, and the first bore 271 in the manifold 10 as pressure and/or suction is applied by a pressure/suction device coupled to the port 220.

In the third position, the secondary sealing portion, illustrated as the land 266, of the seals 260a, 260b, extends over the openings of the second bore 252 of the actuatable valve 250, maintaining a fluid tight seal between the second bore 252 of the actuatable valve 250 and the valve housing 212, thus preventing fluid flow through the second bore 252 and/or fluid bypass between the actuatable valve 250 and the valve housing 212.

While in any one of the first, second and third positions, the primary sealing portions, illustrated as annular sealing members of the seals 260a, 260b, provide a fluid tight seal between the actuatable valve 250 and the housing 212 to prevent fluid leakage or bypass through the interfaces between the actuatable valve 250 and the housing 212 of the manifold 210. In some embodiments, the primary seal portion (e.g., the annular sealing members) may withstand pressures of 300 psi or greater, 400 psi or greater, or 500 psi or greater. The seals 260a, 260b may be placed or seated in the glands 290, 292 of the housing 212 to correctly position and/or prevent backup of the seals 260a, 260b under operative fluid pressures. As shown in the illustrated embodiment, the annuluses of the primary sealing portions of the seals 260a, 260b may be centered on the openings of the first, second, third, and fourth bores 271, 272, 273, 274.

While the valve 250 is at a transitional position intermediate one of the first, second or third positions, the secondary sealing portion of the seal 260a, 260b, illustrated as the lands 264, 265, 266, may provide a fluid tight seal between the valve 250 and the housing 212 to prevent fluid leakage or bypass through the interface between the actuatable valve 250 and the housing 212 of the manifold 210 during actuation of the valve 250, since while in a transitional position intermediate one of the first, second and third positions, the bores 251, 252, 253 of the valve 250 may be misaligned with the openings of the bores 271, 272, 273, 274 of the housing 212.

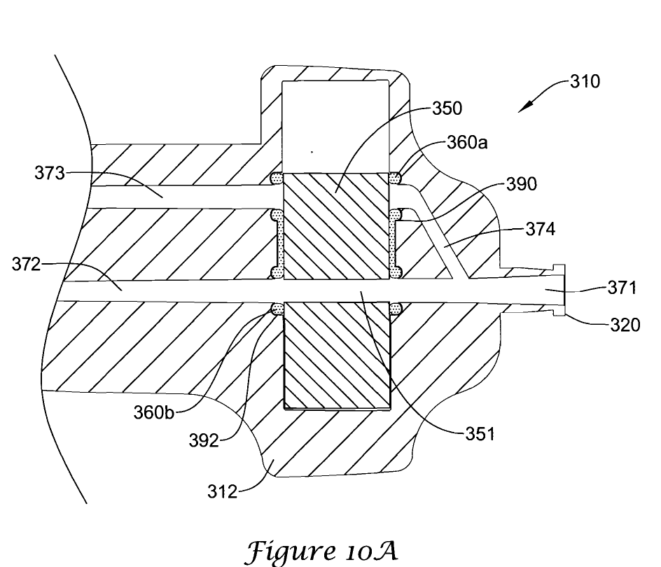

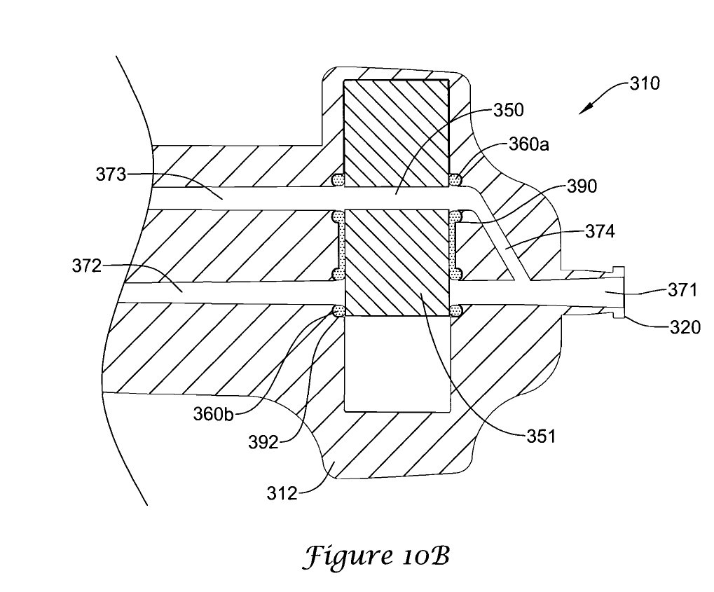

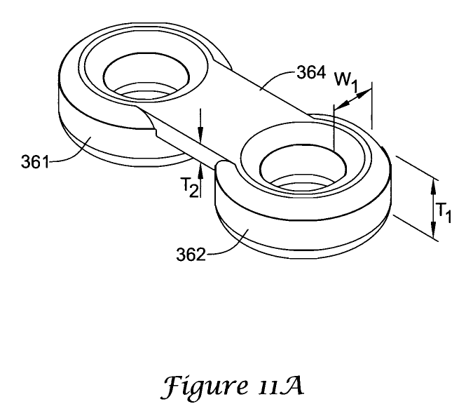

FIGS. 10A and 10B illustrate another alternate embodiment of a manifold 310 including a housing 312 and an actuatable valve 350. As shown in the illustrative embodiment, the actuatable valve 350 may include a bore 351 configured to allow multi-directional fluid flow through the valve 350. The housing 312 includes a plurality of bores, including a first bore 371, a second bore 372, a third bore 373, and a fourth bore 374. Each of the bores 371, 372, 373, 374 may terminate at an opening in the housing 312 juxtaposed to the valve 350.

Additionally, the manifold 310 may include one or more seals for providing a fluid tight seal between the actuatable valve 350 and the housing 312. For example, the manifold 310 may include a first seal 360a and a second seal 360b. The first seal 360a and the second seal 360b may be positioned on opposite sides of the actuatable valve 350, such that when the manifold 310 is in an assembled configuration, the first seal 360a may be positioned between, or interposed between, the actuatable valve 350 and the housing 312. Additionally or alternatively, the second seal 360b may be positioned between, or interposed between, the actuatable valve 350 and the housing 312. Thus, in some embodiments, such as the embodiment illustrated in FIGS. 10A and 10B, the first and second seals 360a, 360b may be positioned in the interfaces between the actuatable valve 350 and the housing 312. In an assembled configuration, the seals 360a, 360b may be compressed between the actuatable valve 350 and the housing 312. In the illustrated embodiment, the first seal 360a and the second seal 360b are substantially the same, thus discussion of only one seal 360 follows. However, in some embodiments, the first seal 360a and the second seal 360b may be dissimilar, each possessing individual characteristics as may be desired for a specific application.

In the illustrated embodiment, the housing 312 includes first and second recessed grooves or glands 390, 392 on opposite sides of the actuatable valve 350 for receiving and seating the seals 360a, 360b. In other embodiments, however, the actuatable valve 350 may include recessed grooves or glands for receiving and seating the seals 360a, 360b.

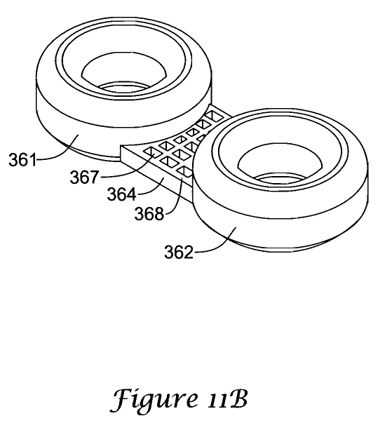

Referring to FIGS. 11A and 11B, the seal 360 may be further described. Similar to the seal 60, the seal 360 may include one or more, or a plurality of sealing elements. For example, the seal 360 may include one, or a plurality of primary sealing portions, such as a first sealing element 361 and a second sealing element 362. Although the seal 360 is shown as including two sealing elements 361, 362, in alternative embodiments the seal 360 may include one, three, four or more sealing elements, such as annular sealing elements. Each of the sealing elements 361, 362 may be any desired shape. For instance, each of the sealing elements 361, 362 may be an annular sealing element resembling an O-ring type seal. A secondary sealing portion, such as a land 364 may connect each of the sealing elements 361, 362 to the adjoining sealing element 361, 362. For example, the land 364 may extend between or bridge the first sealing element 361 with the second sealing element 362. In embodiments including additional sealing elements, additional lands may be included to connect or bridge the additional sealing elements with adjacent sealing elements. In the illustrative embodiment, the first and second sealing elements 361, 362 are aligned along a common axis. In other words, the center of each of the annuluses of the sealing elements 361, 362 lies along a common axis. Likewise, the land 364 of the illustrative embodiment lies along and is centered on the common axis of the seal 360. However, in other embodiments at least one of the sealing elements 361, 362 and/or the land 364 may be misaligned with one or more of the other components of the seal 360.

Similar to the sealing elements 61, 62, 63 of the seal 60, each of the sealing elements 361, 362 may have a thickness, T₁. The thickness, T₁, is intended to describe the distance from one side of the sealing element 361, 362 to the opposite side of the sealing element 361, 362 in a direction parallel to the axial centerline of the opening of the sealing element 361, 362, such as the axial centerline of the central opening of each of the annuluses of the sealing elements 361, 362. Additionally, the land 364 may have a thickness, T₂. The thickness, T₂, of the land 364 is intended to describe the distance in the direction parallel to the thickness, T₁, of the sealing elements 361, 362. In the illustrated embodiment, the thickness, T₁, of the sealing elements 361, 362 is greater than the thickness, T₂, of the land 364. In some embodiments, the thickness, T₂, of the land 364 may be about 75%, about 50%, or about 25% of the thickness, T₁ of the sealing elements 361, 362. However, in other embodiments, the relative thicknesses T₁ and T₂ may be of other proportions. Accordingly, sufficient backup support on the sealing elements 361, 362 may be maintained while being subjected to operative fluid pressures. A larger ratio of T₁ to T₂ (i.e., the higher the T₁/T₂ ratio) leads to more backup support and/or higher pressure capability of the seal. In other embodiments, the thickness, T₁, may be equal to, or substantially equal to the thickness, T₂. In still other embodiments, the thickness, T₂, may be greater than the thickness T₁. As shown in FIG. 11A, one side (the top side) of the land 364 may be a smooth surface and/or be flush with one side (the top side) of the sealing elements 361, 362. However, in other embodiments, the land 364 may be positioned other than flush with the sealing elements 361, 362.

In the illustrative embodiment, the sealing elements 361, 362 may be substantially similar. However, in other embodiments, the first sealing element 361 and the second sealing element 362 may be dissimilar if desired. Similar to the sealing elements 61, 62, 63 of the seal 60, the sealing elements 361, 362 may have an elliptical or oval cross-section. However, in other embodiments, the cross-section of the sealing elements 361, 362 may be circular, non-circular, polygonal, trapezoidal, hexagonal, or any other desired shape. In embodiments where the sealing elements 361, 362 include non-circular or non-regular polygonal cross-sections, the thickness, T₁, of the sealing elements 361, 362 may be chosen to be greater than the width, W₁, of the sealing elements 361, 362. The width, W₁, is intended to describe the distance in the direction perpendicular to the thickness, T₁, across the cross-section of the sealing elements 361, 362. For example, in embodiments wherein the sealing elements 361, 362 are annular sealing elements, the width, W₁, is determined as one half of the difference between the outer diameter and the inner diameter of the annular sealing element. Thus, in embodiments wherein the thickness, T₁, is greater than the width, W₁, the sealing elements 361, 362 may have a greater dimension in the direction of compression than in the transverse direction when the sealing elements 361, 362 are assembled in the housing 312. Among other things, this may alleviate the precision of dimensional tolerances of components of the manifold 310 without compromising the sealing capabilities of the manifold 310.

The land 364 may be described as a band of material connecting adjacent sealing elements 361, 362. Thus, the land 364 may extend between or bridge the sealing elements 361, 362. The land 364 may include a honeycomb webbing defining a plurality of raised portions 368 relative to a plurality of voids 367. The voids 367 may reduce the amount of material necessary to form the land 364. Additionally, the raised portions 368 of the land 364 may contact an adjacent member, be it the actuatable valve 350 or the housing 312. Thus, the honeycomb webbing may reduce the frictional resistance between the land 364 and an adjacent component. Alternatively, a raised portion of the land 364 may include projections, such as a plurality of bumps, knobs, convex surfaces and/or protuberances. In other embodiments, the land 364 may include one or more ribs defining a plurality of raised portions and/or a plurality of voids. Thus, when the land 364 is compressed between the actuatable valve 350 and the housing 312, less than the entire surface area of the bottom side of the land 364 contacts the adjacent surface of the housing 312 and/or the actuatable valve 350, yet maintaining sufficient contact pressure between the sealing surface of the seal 360 and the housing 312. This arrangement may reduce the amount of actuation force necessary to actuate the actuatable valve 350 within the housing 312.

The seal 360 may comprise a material having notable material properties. Some suitable materials may be elastomeric, resilient, inert, biocompatible, compressible and/or incompressible. One suitable material for the seal 360 may be silicone. Other suitable materials for the seal 360 include, but are not limited to, polytetrafluoroethylene (PTFE), polyurethane, neoprene, ethylene propylene, and additional fluoropolymer elastomers.

Again referring to FIGS. 10A and 10B, the opposing sides of the actuatable valve 350 may include annular raised portions which may correspond to the sealing elements 361, 362 as the actuatable valve 350 is positioned in one of multiple positions such that the valve bore 251 is aligned with one or more of the bores 371, 372, 373, 374 of the housing 312. Thus, less than the entire surface of the actuatable valve 350 may be in contact with the seals 360a, 360b in order that the frictional resistance between the seals 360a, 360b and the valve 350 may be reduced without compromising the ability of the seals 360a, 360b to provide a fluid tight seal between the valve 350 and the housing 312.

The functionality of the manifold 310 may now be described while referring to FIGS. 10A and 10B, which illustrate two operational configurations of the manifold 310. The actuatable valve 350 may be actuated between one of multiple positions. For example, in the illustrative embodiment, the valve 350 may be actuated between two positions. The valve 350 may be actuated by sliding the valve 350 between a first position and a second position. In the first position shown in FIG. 10A, the bore 351 of the valve 350 may be aligned with the openings of the first bore 371 and the second bore 372 of the housing 312, defining a fluid pathway therethrough. While in the first position, the valve 350 interrupts the fluid pathway between the third bore 373 and the fourth bore 374. Thus, the bore 351 of the valve 350 may provide multi-directional fluid flow through the valve 350 between the first bore 371 and the second bore 372. In the second position shown in FIG. 10B, the bore 351 of the valve 350 may be aligned with the openings of the third bore 373 and the fourth bore 374 of the housing 312, defining a fluid pathway therethrough. While in the second position, the valve 350 interrupts the fluid pathway between the first bore 371 and the second bore 372. Thus, the bore 351 of the valve 350 may provide multi-directional fluid flow through the valve 350 between the third bore 373 and the fourth bore 374. Thus, the valve 350 may be actuated to select one of multiple pathways through the manifold 310.

In the first position, a pressure/suction device coupled to the port 320 may be used to expel fluid through the fluid pathway defined in the manifold 310 to a medical device, such as a catheter, in fluid communication with the second bore 372. Additionally or alternatively, a pressure/suction device coupled to the port 320 may be used to draw fluid through the fluid pathway defined in the manifold 310 from a medical device, such as a catheter, in fluid communication with the second bore 372 to the pressure/suction device.

While in the first position, the primary sealing portions, illustrated as annular sealing members of the seal 360, provide a fluid tight seal between the actuatable valve 350 and the housing 312 to prevent fluid leakage or bypass through the interfaces between the actuatable valve 350 and the housing 312 of the manifold 310. In some embodiments, the primary seal portion (e.g., the annular sealing members) may withstand pressures of 300 psi or greater, 400 psi or greater, or 500 psi or greater. The seal 360 may be placed or seated in the glands 390, 392 of the housing 312 to correctly position and/or prevent backup of the seal 360 under operative fluid pressures. As shown in the illustrated embodiment, the annuluses of the primary sealing portions of the seal 360 may be centered on the openings of the first, second, third, and fourth bores 371, 372, 373, 374.

The actuatable valve 350 may be actuated between the first position and the second position. For example, the actuatable valve 350 may be slidably actuated between the first position and the second position. In some embodiments, the valve 350 may be actuated between the first position and the second position with an actuation force less than about 4 pounds, less than about 3.5 pounds, or less than about 3.25 pounds. While the valve 350 is at a transitional position intermediate the first position and the second position, the secondary sealing portion of the seal 360, illustrated as the land 364, may provide a fluid tight seal between the valve 350 and the housing 312 to prevent fluid leakage or bypass through the interface between the actuatable valve 350 and the housing 312 of the manifold 310 during actuation of the valve 350, since while in a transitional position intermediate the first position and the second position, the bore 351 of the valve 350 may be misaligned with the openings of the bores 371, 372, 373, 374 of the housing 312.

In the second position, a pressure/suction device coupled to the port 320 may be used to expel fluid through the fluid pathway defined in the manifold 310 to a medical apparatus, such as therapeutic and/or diagnostic substance sources, a contrast media source, a saline source, a waste receptacle, monitoring devices, other fluid sources, receptacles, and/or lines, and the like, in fluid communication with the third bore 373. Additionally or alternatively, a pressure/suction device coupled to the port 320 may be used to draw fluid through the fluid pathway defined in the manifold 310 from a medical apparatus, such as therapeutic and/or diagnostic substance sources, a contrast media source, a saline source, a waste receptacle, monitoring devices, other fluid sources, receptacles, and/or lines, and the like, in fluid communication with the third bore 373 to the pressure/suction device.

While in the second position, the primary sealing portions, illustrated as annular sealing members of the seal 360, provide a fluid tight seal between the actuatable valve 350 and the housing 312 to prevent fluid leakage or bypass through the interfaces between the actuatable valve 350 and the housing 312 of the manifold 310. In some embodiments, the primary seal portion (e.g., the annular sealing members) may withstand pressures of 300 psi or greater, 400 psi or greater, or 500 psi or greater. The seal 360 may be placed or seated in the glands 390, 392 of the housing 312 to correctly position and/or prevent backup of the seal 360 under operative fluid pressures. As shown in the illustrated embodiment, the annuluses of the primary sealing portions of the seal 360 may be centered on the openings of the first, second, third, and fourth bores 371, 372, 373, 374.

The fluid pathways described above which may be established through the illustrative manifolds are intended as exemplary. Those skilled in the art will recognize that additional fluid pathways through the manifolds may be established for a given medical procedure by selectively increasing or decreasing the number of bores and/or ports, and/or modifying selective bores, ports, and/or valve positions to perform a desired function.

Those skilled in the art will recognize that the present invention may be manifested in a variety of forms other than the specific embodiments described and contemplated herein. Accordingly, departure in form and detail may be made without departing from the scope and spirit of the present invention as described in the appended claims.

What is claimed is:

1. A manifold for use in a medical procedure, the manifold comprising:
a port housing having a plurality of ports defined therein;
a valve housing;
an actuatable valve positioned in the valve housing, the actuatable valve adapted for selectively establishing a desired fluid pathway through the manifold; and
a seal interposed between the actuatable valve and the valve housing;
wherein the seal includes a first annular element, a second annular element, and a land extending between the first annular element and the second annular element.

2. The manifold of claim 1, wherein the first and second annular elements have a thickness, and the land has a thickness less than the thickness of the first and second annular elements.

3. The manifold of claim 1, wherein the first and second annular elements each have a thickness and a width, wherein the thickness is greater than the width.

4. The manifold of claim 1, wherein each of the first and second annular elements has an elliptical cross-section.

5. The manifold of claim 1, further comprising a third annular element, and a second land extending between the second annular element and the third annular element.

6. The manifold of claim 1, further comprising a second seal interposed between the actuatable valve and the valve housing opposite the first seal, wherein the second seal includes a first annular element, a second annular element, and a land extending between the first annular element and the second annular element.

7. The manifold of claim 1, wherein the actuatable valve includes a gland for receiving the seal.

8. The manifold of claim 1, wherein the valve housing includes a gland for receiving the seal.

9. The manifold of claim 1, wherein the land includes one or more voids.

10. The manifold of claim 1, wherein the land includes a plurality of raised portions.

11. The manifold of claim 10, wherein the plurality of raised portions includes a plurality of ribs.

12. The manifold of claim 10, wherein the plurality of raised portions includes a plurality of bumps.

13. The manifold of claim 1, wherein the land includes a honeycomb webbing.

14. A manifold for use in a medical procedure, the manifold comprising:
a housing including a plurality of ports and a plurality of bores networking the plurality of ports;
an actuatable valve including one or more bores positioned in the housing, the actuatable valve actuatable between a first position and a second position, such that actuation of the actuatable valve may selectively establish a desired fluid pathway through the manifold; and
a seal interposed between the actuatable valve and the housing;
wherein the seal includes a first sealing element, a second sealing element, and a land extending between the first sealing element and the second sealing element; and
wherein the first and second sealing elements are configured to provide a fluid tight seal between the housing and the actuatable valve when the actuatable valve is in either the first or second position, and the land is configured to provide a fluid tight seal between the housing and the actuatable valve when the actuatable valve is intermediate the first and second positions.

15. The manifold of claim 14, wherein the first sealing element is an annular element and the second sealing element is an annular element.

16. The manifold of claim 14, wherein the first and second sealing elements have a thickness, and the land has a thickness less than the thickness of the first and second sealing elements.

17. The manifold of claim 14, wherein the first and second sealing elements have an elliptical cross-section.

18. The manifold of claim 14, further comprising a second seal interposed between the actuatable valve and the housing opposite the first seal, wherein the second seal includes a first sealing element, a second sealing element, and a land extending between the first sealing element and the second sealing element.

19. A manifold for controlling which of several ports is in fluid communication with a medical device, the manifold comprising:
a housing including a plurality of ports and a plurality of bores networking the plurality of ports;
an actuatable valve having one or more bores selectively in fluid communication with one or more of the plurality of bores of the housing; and
a seal interposed between the housing and the actuatable valve, the seal including a primary seal portion configured to provide a fluid tight seal between the housing and the actuatable valve when the one or more bores of the actuatable valve are aligned with the one or more of the plurality of bores of the housing, and a secondary seal portion configured to provide a fluid tight seal between the housing and the actuatable valve when the one or more bores of the actuatable valve are misaligned with the one or more of the plurality of bores of the housing.

20. The manifold of claim 19, wherein the actuatable valve is actuatable with an actuation force of less than 4 pounds.

21. The manifold of claim 19, wherein the seal can withstand a fluid pressure greater than 400 psi.

22. The manifold of claim 19, wherein the primary seal portion includes one or more annular resilient members having a first thickness, and the secondary seal portion includes a resilient band of material having a second thickness less than the thickness of the one or more annular resilient members.

23. A method of preventing egress of fluid at the interface between two portions of a manifold of a medical device, the method comprising:
providing a manifold housing having a first bore in fluid communication with a first port of the manifold housing and a second bore in fluid communication with a second port of the manifold housing;
providing an actuatable valve slidably actuatable relative to the manifold housing, the actuatable valve including a first bore which may be actuated to be in fluid communication with the first bore of the manifold housing and the second bore of the manifold housing, and the actuatable valve including a second bore which may be actuated to be in fluid communication with the first bore of the manifold housing and the second bore of the manifold housing;
placing a seal between the actuatable valve and the manifold housing, wherein the seal includes a first annular element, a second annular element, and a first land extending between the first annular element and the second annular element.

24. The method of claim 23, wherein the seal is actuatable with the actuating valve, and wherein the first annular element of the seal is aligned with the first bore of the actuatable valve and the second annular element of the seal is aligned with the second bore of the actuatable valve.

25. The method of claim 23, further comprising the step of actuating the actuating valve with an actuation force of less than 4 pounds.
